(19) United States
(12) Patent Application Publication
(10) Pub. No.: US 2021/0378741 A1
(43) Pub. Date: Dec. 9, 2021

(54) ENCLOSED LASER MEDICAL DEVICE/SYSTEM

(71) Applicant: Wolfgang Neuberger, Dubai (AE)

(72) Inventor: Wolfgang Neuberger, Dubai (AE)

(21) Appl. No.: 16/891,656

(22) Filed: Jun. 3, 2020

(51) Int. Cl. A61B 18/22; A61B 90/98

(52) U.S. Cl. CPC A61B 18/22; A61B 2018/00172; A61B 90/98

(57) ABSTRACT

A fiber optic medical device/system is presented, comprising means to connect and enclose a laser source to ensure sterility and safe operation. Device includes a low cost, single use system enclosed and sterilized including laser source and with the fiber attached in a sterile tube. Another embodiment comprises hermetically sealed laser unit with simple connection system to a properly shaped fiber optic, after joining, unit comprises a hand piece and fiber for direct insertion to treat target tissue. Laser handle is aseptically packaged. In other embodiments, fiber is proximally terminated in solid funnel-shaped end to provide unique directional keyed junction with laser module. In another embodiment, unit includes fiber proximally joined to it, where a laser module can be added away from the fiber end, so that laser does not need to be sterilized but entire unit is safe for medical use in sterile fields. Enclosure also serves as a grip for pulling and/or manipulating device. In another preferred embodiment, laser unit is attached to a rigid delivery system for laparoscopic procedures.

### CROSS REFERENCE TO RELATED APPLICATION

This patent application is a division of, and claims priority to, co-pending U.S. patent application Ser. No. 14/767,548 filed Aug. 12, 2015, entitled “Enclosed Laser Medical Device/System”, which is hereby expressly incorporated in its entirety by reference as part of the present disclosure.

### BACKGROUND OF THE INVENTION

### 1. Field of the Invention

The present invention relates to minimally invasive medical treatment systems and to in particular, to medical treatments/systems using self-contained energy emitting devices and conveying means.

### 2. Prior Art Disclosure Statement

Laser systems can operate as beneficial and effective medical instruments. They allow specific treatment to be administered with minimal invasiveness. Laser treatments are frequently preferred by those skilled in the art in different applications. There is an increasing number of medical applications involving the use of laser devices in a wide range of specialized disciplines such as angiology, proctoscopy, otolaryngology, urology, gynecology and aesthetics. Worth specific mention are treatment of insufficient veins, benign hyperplasic prostate (BPH), hemorrhoids, ulcers, fistulas, calculi, and ear, nose and throat (ENT) disorders just to name a few.

Minimally invasive laser treatments have been improved due to new diode laser systems. Diode laser systems are greatly advantageous in comparison with other existing laser source technologies such as CO2, holmium:YAG, pulsed-dye, Nd:YAG or KTP laser sources, in that they provide higher output, at reduced dimensions and weight. They also have simpler and smaller air cooling systems. Moreover, being integrated with optical fibers, they have a high reliability and do not need alignment. In summary, they provide great efficiency and robustness.

Consequently there are also an increasing number of different scenarios in which laser devices are needed, ranging from the physician's office to high tech operating rooms. Moreover many single laser devices are unspecific enough so that one same device is optimal for more than one type of treatment. In many cases, the same laser device can be used in the operating room for aesthetic surgery, within a well-equipped vascular surgeon's office or in a simpler otolaryngologist's office. However, when all of these scenarios exist in one healthcare center, there is at least one of these medical devices assigned to each treatment application, since their size, weight and complexity may not justify a logistics system to rotate one device among the different areas. Furthermore, the size of existing equipment requires a certain storage space that may not be available in every health unit in which it is used. Additionally, the number of cables involved in connecting equipment make using the device complex and uncomfortable and in some cases dangerous. Finally, in some cases, equipment belongs to physicians, not to the health care center where they work, and because of size and weight it is difficult for them to take equipment with them to, for example other offices or healthcare centers.

Thus, medical device designers have made attempts to address some of mentioned drawbacks. Below are some examples of such attempts.

In US Patent Publication 2008/0077198 by Webb et al, a handheld self-contained nerve-stimulation device using light to provide a source of stimulation on one or more nerve fibers is disclosed. Stimulation is provided through a device wherein a laser or led light source is mounted to the hand piece. Light is passed from the light source through optical tip to stimulate nerves. The hand piece contains a self-contained power source, such as batteries. US Application 2010/0106146 by Boitor et al. discloses a handheld portable diode laser surgical device that includes a power supply, a laser diode, integral control interface and display, and a multicomponent sterile, disposable tip apparatus featuring assembly for alignment of a self-contained optical fiber to the surgical device, and releasable locking assembly between the tip apparatus and surgical device. An embodiment includes wireless foot pedal on/off control and a dock providing sterile, antiseptic recharging environment. US Patent Application 2009/0275930 by Di Sessa et al. disclose a multi-component sterile, disposable tip apparatus for laser surgical devices. It features assembly for alignment of a self-contained optical fiber to the surgical device and releasable locking assembly between the tip and device. U.S. Pat. No. 6,724,958 by German et al. discloses an invention that is a self-contained handheld surgical laser that provides surgeons with the optimal laser beam parameters for laser tissue soldering. The laser is incorporated in a battery powered self-contained handheld apparatus with a roughly uniform distribution of light intensity across the beam. The laser apparatus provides laser energy at a wavelength absorbed by the chromophore utilized in the laser solder with optimum parameters for tissue soldering.

Mentioned existing prior art possesses several limitations. First, these are very low power units. Output energy emitted from devices is insufficient or ineffective for several common medical applications. Secondly, laser source is not presented as sterile. Additionally, mentioned small-sized prior art does not consider easy maneuverability, an important issue in many delicate medical applications. Finally, none of mentioned devices gather all the features described above in a single sterile device, namely, small size, low cost, cable-less, and easy to handle. Consequently, formerly disclosed compact-sized devices are difficult to maneuver, comprising discomfort for the physician and a potential hazard for the patient.

There is thus, a need for a safe, compact, low cost, disposable medical laser treatment device/system that is versatile and easy to maneuver. The present invention addresses these needs.

### Objectives and Brief Summary of the Invention

It is an objective of the present invention to provide a device and method for treatment of different medical conditions.

It is another objective of the present invention to treat several types of medical conditions by using a single localized, directed energy source and conveying means.

It is yet another objective of the present invention to provide a portable and versatile sterile laser medical device/system.

It is still another objective of the present invention to provide medical laser device that is safe and easily maneuverable.

Briefly stated, a fiber optic medical device/system is presented, comprising means to connect and enclose a laser source to ensure sterility and safe operation. Device includes a low cost, single use system enclosed and sterilized including laser source and with the fiber attached in a sterile tube. A preferred embodiment comprises hermetically sealed laser unit with simple connection system to a properly shaped short fiber optic, where, after joining, unit comprises a hand piece and fiber for direct insertion to treat target tissue. Laser handle is aseptically packaged. In other embodiments, fiber is proximally terminated in solid funnel-shaped end to provide unique directional keyed junction with laser module. In another embodiment, unit includes fiber proximally joined to it, where a laser module can be added away from the fiber end, so that laser does not need to be sterilized but entire unit is safe for medical use in sterile fields. Enclosure also serves as a grip for pulling and/or manipulating device. In another preferred embodiment, laser unit is attached to a rigid delivery system for laparoscopic procedures.

The above and other objects, features and advantages of the present invention will become apparent from the following description read in conjunction with the accompanying drawings (in which like reference numbers in different drawings designate the same elements).

### BRIEF DESCRIPTION OF FIGURES

FIG. 1 depicts a preferred embodiment of present invention describing main components of the system disclosed.

FIG. 2 shows different preferred connection systems between laser device and conveying means.

FIG. 3 depicts a sketch of a preferred embodiment of present invention.

FIG. 4 shows another preferred embodiment with a complete disposable unit with rigid delivery end.

### DETAILED DESCRIPTION OF PREFERRED EMBODIMENTS

The present invention addresses prior art disadvantages by providing a portable, cable-less, disposable laser device for carrying out a number of different medical treatments while assuring sterility.

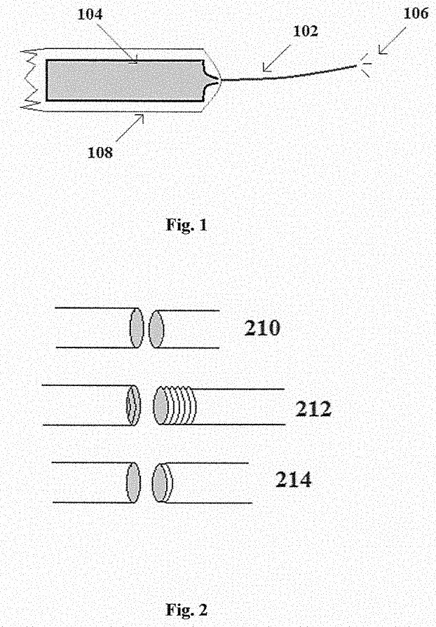

In a preferred embodiment, depicted in FIG. 1, system comprises a fiber optic medical treatment device 102 attached to source 104, which is kept within sterile enclosure 108. Optical fiber's firing end 106 emits radiation at target tissue by means of a variety of possible radiation patterns according to firing end including but not limited to off-axis firing end, a side-firing distal end, a radial emitting end or a direct emitting end. When laser radiation is used to apply energy to the vessel, different wavelengths can be chosen. Laser wavelength is chosen, in the present case, according to the desired penetration depth in tissue and desired effect in tissue. Radiofrequency, microwave, thermal and other energy sources may be used to reliably and controllably perform the task and the method described, provided suitable enhancers and/or imaging means as described are used.

In a preferred embodiment, system comprises hermetically sealed laser unit with simple hook up system to an optical fiber. Hook up systems, as shown in FIG. 2 include but are not limited to plug-socket (male-female), 210, screw-on 212, or snap-on 214 connectors. Such hook up systems can be stereo-specific to provide a specific orientation of the fiber distal tip relative to hand piece. Once fiber optic is connected, unit commands a hand piece and fiber for direct insertion into target tissue. Laser handle is aseptically packaged. In preferred embodiments, fiber is proximally terminated in solid funnel-shaped end to provide unique directional keyed junction with laser module. In another preferred embodiment, unit includes fiber proximally joined to it, where a laser module can be added away from the fiber end. This way laser does not need to be sterilized and entire unit is safe for medical use in sterile fields. In another embodiment, outer contour of laser module has a grooved pattern to permit easy grip. This way, enclosure also serves as a grip for making maneuvers with device/system for example a pull-back movement during insufficient vein treatments; twisting motion for urologic treatments or back-and-forth movements in liposuction techniques.

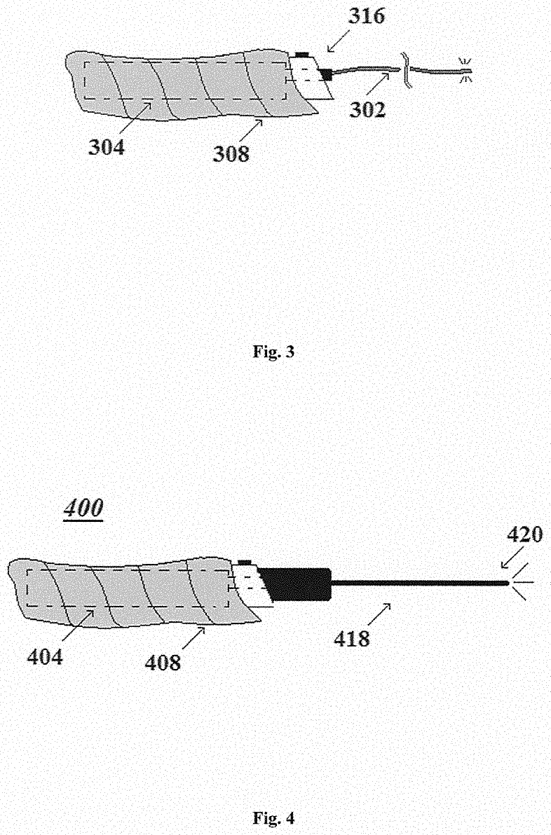

An example of a preferred embodiment is a small-sized device with an ergonomic handgrip for portable handheld applications. FIG. 3 shows a sketch of preferred embodiment. Energy source 304 is a 5 Watt 1470 nm battery driven diode laser that conveys energy through a 30-40 cm long optical fiber 302. Alternatively, for higher energy applications, energy source 304 is an electrically driven 20-Watt diode laser. Enclosure 308 also plays the role of a handgrip. System includes an RFID technology system 316 to identify the laser/fiber package and/or permit activation for specific parameters such as power level, energy and density. This eliminates the need for cables that make handling medical devices difficult, uncomfortable and sometimes even dangerous.

In another preferred embodiment, as depicted in FIG. 4, a complete disposable unit 400 includes a laser source 404 enclosed within ergonomic handgrip 408 attached to a rigid conveying means 418 and delivery end 420 for more complex surgical procedures such as laparoscopic procedures. In another embodiment, such unit is handled via remote control for complex for robotic surgeries.

Embodiments in this invention present several advantages. One advantage is its potentially small size. Another advantage is its versatility for several different application configurations. It is thus, a useful tool for health care settings that carry out various different laser treatments. This possibility allows the health care center to be more efficient and to reduce costs. Additionally, because it can be commanded by RFID technology and system is battery operated, cables are not needed. This contributes also to its versatility feature. Finally, disclosed invention is low in cost and is therefore feasible for consideration as a practical, compact, low cost, disposable device in many health care settings, such as in different hospital care units and specific independent treatment offices.

Having described preferred embodiments of the invention with reference to the accompanying drawings, it is to be understood that the invention is not limited to the precise embodiments, and that various changes and modifications may be effected therein by skilled in the art without departing from the scope or spirit of the invention as defined in the appended claims.

What is claimed is:

1. A fiber optic medical treatment device/system comprising: a laser energy source; an enclosure; an optical fiber; and means to connect and enclose a laser source to ensure sterility and provide safe operation

2. The fiber optic medical treatment system according to claim 1, wherein said device is disposable, i.e. a single use device.

3. The fiber optic medical treatment system according to claim 1, wherein said laser source is hermetically sealed in said enclosure.

4. The fiber optic medical treatment device/system according to claim 1, further comprising a stereo-specific connection system to said optical fiber.

5. The medical treatment device/system according to claim 4, wherein said optical fiber comprises a funnel-shaped proximal end for a directional keyed junction with said laser energy source unit.

6. The medical treatment device/system according to claim 1, wherein said enclosure serves as a handgrip for manipulating said device.

7. The medical treatment device/system according to claim 1, wherein said laser energy source unit is battery-powered.

8. The medical treatment device/system according to claim 1, further comprising RFID technology to identify laser/fiber package.

9. The medical treatment device/system according to claim 1, further comprising RFID technology to control radiation parameters.

10. A medical treatment device/system comprising: a laser energy source connected to a rigid laser conveying means enclosed to ensure sterility and provide safe operation.

11. The medical treatment system according to claim 10, wherein said device is disposable, a single use device.
